(19) United States
(12) Patent Application Publication
(10) Pub. No.: US 2012/0039966 A1
(43) Pub. Date: Feb. 16, 2012

(54) Rinse-Off Care Products and Consumer Product Line-ups Comprising Same

(76) Inventors: Amy Michelle Capretta, Cincinnati, OH (US); Melissa Ann Kreuzer, Fairfield, OH (US); Anthony Charles Lanzalaco, Fairfield, OH (US)

(21) Appl. No.: 12/854,985

(22) Filed: Aug. 12, 2010

(51) Int. Cl. A61K 8/02; A61K 8/27; A61Q 15/00; A61K 8/18

(52) U.S. Cl. 424/401; 424/65; 424/67

(57) ABSTRACT

Consumer product line-ups are provided that include a rinse-off care product that may be used in conjunction with a traditional leave-on antiperspirant or deodorant product to better manage underarm malodor.

### FIELD OF THE INVENTION

The present invention is directed to rinse-off products containing particulate an antimicrobial agent for use on targeted body parts, such as the underarm, to manage malodor. The present invention is also directed to consumer product line-ups that include a leave-on antiperspirant or deodorant product and a rinse-off care product. The present invention also provides methods for managing malodor associated with specific body parts.

### BACKGROUND OF THE INVENTION

Commercial products that are specifically targeted for underarm application include various forms of antiperspirant and deodorant products. These products are typically applied as leave-on products, in that the area of skin to which they are applied is not rinsed directly following application. For some consumers, the commercially available antiperspirant and deodorant products satisfactorily control underarm malodor. For others, a single daily application of an antiperspirant or deodorant product cannot adequately curb underarm malodor. And still others do not want to run the risk of a malodor breakthrough during the day. Accordingly, there is a need for additional/alternative underarm products, treatments and/or regimens.

### SUMMARY OF THE INVENTION

The present invention is directed to consumer product line-ups that include a rinse-off care product that may be used in conjunction with a traditional leave-on antiperspirant or deodorant product to better manage underarm malodor. As used herein, the term “rinse-off” means the intended product usage includes application to skin followed by rinsing of the skin within a few seconds or minutes of the application step. The rinse-off care product can be specifically marketed as a product that may be used on targeted body parts as opposed to an all over body wash; for example, for use on the underarm, groin, feet, or any combination thereof. In this regard, the rinse-off care products may contain indicia to serve as a product selection guide to help consumers make an informed purchase decision towards meeting their particular desires or needs.

When designed for underarm application, the rinse-off care products may be positioned near or within the shelving area comprising leave-on antiperspirant and deodorant products so that consumers desiring an additional underarm care option can readily locate and purchase the same. The shelving proximity can also educate consumers about the possibility of an underarm treatment regimen that includes the use of a rinse-off care product followed by a leave-on antiperspirant or deodorant product.

### DETAILED DESCRIPTION OF THE INVENTION

The present invention may be understood more readily by reference to the following detailed description of illustrative and preferred embodiments. It is to be understood that the scope of the claims is not limited to the specific components, methods, conditions, devices, or parameters described herein, and that the terminology used herein is not intended to be limiting of the claimed invention. Also, as used in the specification, including the appended claims, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Similarly, when values are expressed as approximations, by use of the antecedent basis “about,” it will be understood that the particular values form another embodiment. All ranges are inclusive and combinable.

All percentages, parts and ratios are based upon the total weight of the products of the present invention, unless otherwise specified. All such weights as they pertain to listed ingredients are based on the active level and, therefore, do not include carriers or by-products that may be included in commercially available materials. Except as otherwise noted, all amounts including quantities, percentages, portions, and proportions, are understood to be modified by the word “about”, and amounts are not intended to indicate significant digits. Except as otherwise noted, the articles “a”, “an”, and “the” mean “one or more”

The products and methods/processes of the present invention can comprise, consist of, and consist essentially of the essential elements and limitations of the invention described herein, as well as any of the additional or optional ingredients, components, steps, or limitations described herein.

The present invention includes embodiments directed to rinse-off care products that can be applied to specific body parts to manage malodor associated with the same. The present invention also includes embodiments directed to product line-ups that contain, inter alia, a leave-on antiperspirant or deodorant product and a rinse-off care product. Exemplary antiperspirant/deodorant and rinse-off care products will first be described, followed by a description of exemplary line-up positioning of the various products to facilitate selection and purchase of a leave-on antiperspirant/deodorant product and a rinse-off care product to be used according to a treatment regimen.

### Exemplary Leave-on Antiperspirant/Deodorant Products

Leave-on antiperspirant and deodorant products typically comprise a carrier material. Suitable carriers include, but are not limited to, any topically safe and effective organic, silicone-containing or fluorine-containing, volatile or non-volatile, polar or non-polar liquid carrier. Exemplary carriers are liquid under ambient conditions, and can include one or more liquid carrier materials provided that the any such combination of materials is in liquid form under ambient conditions. Depending on the type of product form desired, concentrations of the liquid carrier in the products will typically range from about 10% or from about 30% to about 90% or to about 75%, by weight of the product. The antiperspirant/deodorant products may be formulated as an aqueous or anhydrous product. Aqueous products may comprise from about 10% or from about 15% water, by weight of the product to about 75%, to about 60%, or to about 50% water, by weight of the product. Anhydrous products may comprise less than about 10%, less than about 3%, less than about 1%, or zero percent water, by weight of the product.

The antiperspirant/deodorant products typically also include an active. By way of example only, the antiperspirant actives may include astringent metallic salts, especially inorganic and organic salts of aluminum, zirconium and zinc, as well as mixtures thereof. A representative, non-limiting list of suitable deodorant actives includes ethylenediaminetetraacetic acid, cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmethyl sarcosine, lauroyl sarcosine, N-myristoyl glycine, potassium N-lauryl sarcosine, trimethyl ammonium chloride, sodium aluminum chlorohydroxy lactate, triethyl citrate, tricetylmethyl ammonium chloride, 2,4,4′-trichlorio-2′-hydroxy diphenyl ether (triclosan), 3,4,4′-trichlorocarbanilide (triclocarban), L-lysine hexadecyl amide, zinc pyrithione, zinc phenolsulfate, farnesol, and mixtures thereof.

Exemplary antiperspirant/deodorant products may also comprise thickening agents to help provide the product with the desired viscosity, rheology, texture and/or product hardness, or to otherwise help suspend any dispersed solids or liquids within the product. The term “thickening agent” may include any material known or otherwise effective in providing suspending, gelling, viscosifying, solidifying or thickening properties to the product or which otherwise provide structure to the final product form. These thickening agents may include gelling agents, polymeric or nonpolymeric agents, inorganic thickening agents, or viscosifying agents. The thickening agents may include organic solids, silicone solids, crystalline or other gellants, inorganic particulates such as clays or silicas, or combinations thereof. The concentration and type of the thickening agent selected for use in the antiperspirant/deodorant product will vary depending upon the desired product form, viscosity, and hardness. The thickening agents suitable for use herein, may have a concentration range from at least about 0.1%, at least about 3%, or at least about 5% but no more than about 35%, no more than about 20%, or no more than about 10%, by weight of the product. Non-limiting examples of suitable gelling agents include fatty acid gellants, salts of fatty acids, hydroxyl acids, hydroxyl acid gellants, esters and amides of fatty acid or hydroxyl fatty acid gellants, cholesterolic materials, dibenzylidene alditols, lanolinolic materials, fatty alcohols, triglycerides, sucrose esters such as SEFA behenate, inorganic materials such as clays or silicas, other amide or polyamide gellants, and mixtures thereof.

The antiperspirant/deodorant products may further comprise one or more optional components which may modify the physical or chemical characteristics of the products or serve as additional “active” components when deposited on the skin. Of course, such optional components may be included provided that they are physically and chemically compatible and do not otherwise unduly impair product stability, aesthetics, or performance. Nonlimiting examples of such optional materials include, but are not limited to, pH buffering agents, malodor controlling agents, fragrance materials, emollients, humectants, soothing agents, dyes and pigments, medicaments, baking soda and related materials, preservatives, and soothing agents such as aloe vera, allantoin, D-panthenol, pantothenic acid derivatives (e.g., those disclosed in U.S. Pat. No. 6,495,149), avocado oil and other vegetative oils, and lichen extract.

The antiperspirant/deodorant products may be made in a variety of forms including, for example, solid, soft solid, spray, roll-on, and aerosol. Aerosol products employ a propellant in the product. Exemplary propellants include dimethyl ether, carbon dioxide, nitrous oxide, 1,1 difluoroethane, 1,1,1,2 tetrafluoro ethane, butane, isobutane, pentane, isopentane, propane, and mixtures thereof.

### Exemplary Rinse-Off Care Products

Exemplary rinse-off care products include an aqueous carrier, which can be present at a level of from about 20% to about 95%, or from about 60% to about 85%. The aqueous carrier may comprise water, or a miscible mixture of water and organic solvent. Non-aqueous carrier materials may also be employed.

The rinse-off care products may include one or more detersive surfactants. The detersive surfactant component is included to provide cleaning performance to the product. The detersive surfactant component in turn comprises anionic detersive surfactant, zwitterionic or amphoteric detersive surfactant, or a combination thereof. A representative, non-limiting, list of anionic surfactants includes anionic detersive surfactants for use in the products include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. In a further embodiment of the present invention, the anionic surfactant is preferably sodium lauryl sulfate or sodium laureth sulfate. The concentration of the anionic surfactant component in the product should be sufficient to provide a desired cleaning and/or lather performance, and generally ranges from about 2% to about 50%.

Amphoteric detersive surfactants suitable for use in the rinse-off care products are well known in the art, and include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate.

Zwitterionic detersive surfactants suitable for use in the rinse-off care products are well known in the art, and include those surfactants broadly described as derivatives of aliphatic quaternaryammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate.

The rinse-off care products may further comprise additional surfactants for use in combination with the anionic detersive surfactant component described hereinbefore. Non-limiting examples of other anionic, zwitterionic, amphoteric or optional additional surfactants suitable for use in the products are described in McCutcheon's, Emulsifiers and Detergents, 1989 Annual, published by M. C. Publishing Co., and U.S. Pat. Nos. 3,929,678, 2,658,072; 2,438,091; and 2,528,378.

Exemplary rinse-off care products can employ a particulate antimicrobial agent. The antimicrobial agent and carrier material (and/or other solvent-acting material ingredients) should be chosen such that the antimicrobial agent remains as a solid particulate within the final formulation and upon application to the skin; that is, the antimicrobial agent is not completely solubilized prior to use. Remaining in particulate form within the final formulation and upon application to the skin enables at least a portion of the antimicrobial agent to deposit into skin crevices and/or hair follicles, and to survive rinsing. Microscopy can enable one to determine the presence of discrete antimicrobial agent particles within the final formulation. In some embodiments, the antimicrobial agent particles are substantially insoluble in the final composition; that is, less than 5% or 1% by weight of the particle being dissolved based on the chosen antimicrobial agent and solvent(s) employed in the final rinse-off formulation.

The antimicrobial agent particles generally range in particle size from about 0.5 μm to about 100 μm, and preferably from about 0.5 μm to about 50 μm. It should be appreciated that not all of the plurality of antimicrobial agent particles within a given product necessarily falls within the above range and that the particle distribution may be normal or not. It is believed that antimicrobial particles in the 0.5 μm to 10 μm size range can deposit into hair follicles, while larger particles (e.g., 10-100 μm or even larger) may deposit into skin folds, wrinkles, or other surface irregularities that can be present in malodor harboring body parts, such as the underarm. Particles that are larger than 100 μm may be perceptible during application and impart a gritty feel. Some consumers may desire the tactile feedback with larger particles, believing that the grittiness is helping to removed unwanted material from the skin; thus, some embodiments of the present invention employ at least some particles larger than 100 μm. In other embodiments, antimicrobial particles larger than 100 μm are minimized or absent altogether.

Staphylococcus epidermidis and corynebacterium mucifaciens are two of the key odor-causing bacteria associated with the human underarm. Accordingly, when the rinse-off care products are designed for managing underarm malodor, the antimicrobial agent preferably has a Minimal Inhibitory Concentration (“MIC”) of less than or equal to 2,500 μg/ml, 1,000 μg/ml, 500 μg/ml, or 100 μg/ml against at least one strain of at least one of these bacteria. MIC values can be obtained using a traditional broth dilution microbiological technique, such as that described in the following journal article: Andrews, J. M., “Determination of minimum inhibitory concentrations”, Journal of Antimicrobial Chemotherapy 48 (supl. 1): 5-16, 2001.

Suitable anti-microbial agents can include, but are not limited to, metals (e.g., Zn, Cu, Al, Ti, Sn, Bi, and Ag), metal salts (e.g., zinc carbonate, copper sulfate, and zinc gluconate), metal pyrithione salts (e.g., ZPT and CuPT), zeolites, metal zeolites, quaternary ammonium compounds (e.g., cetyl pyridinium chloride, and benzylalkonium chloride), quat bound clays, metal bound clays, and PolyAspirin (e.g., as described in PCT publication no. WO 2008/034019). The antimicrobial agent can be employed in the rinse-off care products at levels of from about 0.1% to about 5%; other levels may however also be possible.

In one exemplary embodiment, the rinse-off care product contains a zinc-containing antimicrobial agent. Such agents can include, for example, a zinc salt of 1-hydroxy-2-pyridinethione (known as “zinc pyrithione” or “ZPT”); more preferably ZPT in platelet particle form.

Zinc-containing layered structures can also be employed, and are typically those materials with crystal growth primarily occurring in two dimensions. It is conventional to describe layer structures as not only those in which all the atoms are incorporated in well-defined layers, but also those in which there are ions or molecules between the layers, called gallery ions (A. F. Wells “Structural Inorganic Chemistry” Clarendon Press, 1975). Zinc-containing layered materials (ZLM's) may have zinc incorporated in the layers and/or be components of the gallery ions.

Many ZLM's occur naturally as minerals. Common examples include hydrozincite (zinc carbonate hydroxide), basic zinc carbonate, aurichalcite (zinc copper carbonate hydroxide), rosasite (copper zinc carbonate hydroxide) and many related minerals that are zinc-containing. Natural ZLM's can also occur wherein anionic layer species such as clay-type minerals (e.g., phyllosilicates) contain ion-exchanged zinc gallery ions. All of these natural materials can also be obtained synthetically or formed in situ in a product or during a production process.

Another common class of ZLM's, which are often, but not always, synthetic, is layered doubly hydroxides, which are generally represented by the formula [M²⁺₁₋ₓM³⁺ₓ(OH)₂]^{x+}A^{m-}_{x/m}.nH₂O and some or all of the divalent ions (M²⁺) would be represented as zinc ions (Crepaldi, E L, Pava, P C, Tronto, J, Valim, J B J. Colloid Interfac. Sci. 2002, 248, 429-42).

Yet another class of ZLM's can be prepared called hydroxy double salts (Morioka, H., Tagaya, H., Karasu, M, Kadokawa, J, Chiba, K Inorg. Chem. 1999, 38, 4211-6). Hydroxy double salts can be represented by the general formula [M²⁺₁₋ₓM²⁺₁₊ₓ(OH)_{3(1-y)}]⁺Aⁿ⁻_{(1=3y)/n}.nH₂O where the two metal ion may be different; if they are the same and represented by zinc, the formula simplifies to [Zn₁₊ₓ(OH)₂]^{2x+}2xA⁻.nH₂O. This latter formula represents (where x=0.4) common materials such as zinc hydroxychloride and zinc hydroxynitrate. These are related to hydrozincite as well wherein a divalent anion replace the monovalent anion. These materials can also be formed in situ in a product or in or during a production process.

These classes of ZLM's represent relatively common examples of the general category and are not intended to be limiting as to the broader scope of materials which fit this definition.

Commercially available sources of basic zinc carbonate include Zinc Carbonate Basic (Cater Chemicals: Bensenville, Ill., USA), Zinc Carbonate (Shepherd Chemicals: Norwood, Ohio, USA), Zinc Carbonate (CPS Union Corp.: New York, N.Y., USA), Zinc Carbonate (Elementis Pigments: Durham, UK), and Zinc Carbonate AC (Bruggemann Chemical: Newtown Square, Pa., USA).

Basic zinc carbonate, which also may be referred to commercially as “Zinc Carbonate” or “Zinc Carbonate Basic” or “Zinc Hydroxy Carbonate”, is a synthetic version consisting of materials similar to naturally occurring hydrozincite. The idealized stoichiometry is represented by Zn₅(OH)₆(CO₃)₂ but the actual stoichiometric ratios can vary slightly and other impurities may be incorporated in the crystal lattice.

Some of the rinse-off care product embodiments of the present invention include a combination of ZPT and a ZLM (such as, for example, basic zinc carbonate).

The rinse-off care products may further comprise one or more optional components known for use in personal care products, provided that the optional components are physically and chemically compatible with the essential components described herein, or do not otherwise unduly impair product stability, aesthetics or performance. Individual concentrations of such optional components may range from about 0.001% to about 10%. Non-limiting examples of optional components for use in the product include cationic polymers, conditioning agents (hydrocarbon oils, fatty esters, silicones), suspending agents, viscosity modifiers, dyes, nonvolatile solvents or diluents (water soluble and insoluble), pearlescent aids, foam boosters, additional surfactants or nonionic cosurfactants, pediculocides, pH adjusting agents, perfumes, preservatives, chelants, proteins, skin active agents, sunscreens, UV absorbers, and vitamins, minerals, herbal/fruit/food extracts, sphingolipids derivatives or synthetical derivative, and clay.

Indicia may be associated with the rinse-off care product. The indicia can indicate or communicate various matters to consumers. For example, the indicia may serve as a product selection guide through indicating indicium that the rinse-off product is for a targeted body part (e.g., underarm) and/or that the rinse-off care product is used in combination with a second product (e.g., a leave-on antiperspirant or deodorant product) to manage malodor.

The indicia may be included on a printed substrate. The printed substrate can be employed as part or all of the primary and/or secondary packaging of the rinse-off care product.

“Primary packaging,” as used herein, means any container in which the care product is in direct contact. And “secondary packaging” means any additional materials that are associated with the primary packaging, such as, for example, a box or polymeric sleeve that at least partially surrounds the primary packaging. The printed substrate may also be a brochure, instruction sheet, or other informative document that is associated with the rinse-off care product. Although not required by the present invention, these documents are typically included in or attached (permanently or temporarily) to packaging materials.

Advertisement is another exemplary method of communicating product information to perspective buyers through associated indicia. Illustrative advertisement media includes television/internet broadcasts, a page in a magazine or newspaper, a sticker, a coupon, a flyer, an in-aisle or end-of-aisle display, and point-of-sale items intended to either be taken by prospective buyers or remain in an area proximate the rinse-off care product. It should be noted that if advertisement which is disconnected from the rinse-off care product itself is utilized, then the communication provided in the advertisement may or may not be reproduced on packaging of the care product.

In accordance with one exemplary embodiment, the rinse-off care product includes (a) a container; (b) a composition disposed within the container, the composition comprising a liquid carrier material and an antimicrobial agent, the antimicrobial agent being in the form of solid particles within the composition, at least some of the solid particles being sized for delivery into underarm hair follicles; (c) a first indicium associated with the container that serves as a product selection guide through an indication that the rinse-off care product is for underarm application to manage body odor; and (d) a second indicium associated with the container that instructs a user to apply the composition to an underarm, followed by a rinse step.

In accordance with another exemplary embodiment, the rinse-off care product includes a (a) container; (b) a composition disposed within the container, the composition comprising a liquid carrier material and an antimicrobial agent, the antimicrobial agent being in the form of solid particles within the composition and having an MIC value of 2500 μg/ml or less against at least one strain of a least one of staphylococcus epidermidis and corynebacterium mucifaciens; (c) a first indicium associated with the container that serves as a product selection guide through an indication that the rinse-off care product is for underarm application to manage body odor; and (d) a second indicium associated with the container that instructs a user to apply the composition to an underarm, followed by a rinse step.

Another exemplary rinse-off care product includes (a) a container; (b) a composition disposed within the container, the composition comprising a liquid carrier material and an antimicrobial agent; (c) a first indicium associated with the container that serves as a product selection guide through an indication that the rinse-off care product is for underarm application to manage body odor; (d) a second indicium associated with the container that instructs a user to apply the composition to an underarm, followed by a rinse step; and (e) a third indicium associated with the container that indicates application of a leave-on antiperspirant or deodorant product after the rinse-off care product is used.

In accordance with yet another exemplary embodiment, the rinse-off care product includes (a) a container; (b) a composition disposed within the container, the composition comprising a liquid carrier material and an antimicrobial agent; (c) a first indicium associated with the container that serves as a product selection guide through an indication that the rinse-off care product is for underarm application to manage body odor; (d) a second indicium associated with the container that instructs a user to apply the composition to an underarm, followed by a rinse step; and (e) a third indicium associated with the container that indicates that the rinse-off care product can be used in place of a leave-on antiperspirant or deodorant product.

The rinse-off care product may be applied by a variety of means, including by rubbing, wiping or dabbing with hands or fingers, or by means of an implement and/or delivery enhancement device. Non-limiting examples of implements include a sponge or sponge-tipped applicator, a mesh shower puff, a swab, a brush, a wipe, and combinations thereof. Non-limiting examples of delivery enhancement devices include mechanical, electrical, ultrasonic and/or other energy devices. Employment of an implement or device may help delivery of the particulate antimicrobial agent to target regions, such as, for example, hair follicles and undulations that can exist in the underarm. The rinse-off care product may be sold together with such an implement or device. Alternatively, an implement or device can be sold separately but contain indicium to indicate usage with a rinse-off care product. Implements and delivery devices can employ replaceable portions (e.g., the skin interaction portions), which can be sold separately or sold together with the rinse-off care product in a kit.

### Exemplary Consumer Product Line-Ups

The consumer product line-ups of the present invention including positioning the rinse-off care products at various positions relative to the leave-on antiperspirant and deodorant products to help communicate to prospective purchasers that a rinse-off product targeted to specific parts of the body (e.g., the underarm, groin region, feet) is available and may be used together with a leave-on antiperspirant or deodorant to manage malodor. Traditional all over body washes and other hygiene products are typically shelved at locations that are separate from the antiperspirant and deodorant products, and thus, prospective buyers may not readily appreciate that two or more underarm products can be used in conjunction with one another to manage malodor. In that regard, the rinse-off care products may contain packaging indicium (e.g., same brand names, artwork, instructions, and/or same manufacturer) that communicates application of a leave-on antiperspirant or deodorant product after application of the rinse-off care product. Positioning a rinse-off care product near or within the leave-on antiperspirant and deodorant products can also communicate that an alternative product, as compared to a traditional leave-on underarm product, may be used on the underarm to manage malodor.

In accordance with one exemplary embodiment, the consumer product line-up comprises a plurality of different leave-on antiperspirant and/or deodorant products positioned within a first shelving area, a plurality of different body wash products positioned with a second shelving area that is separate and distinct from the first shelving area, and a rinse-off care product positioned on shelving that is closer to the first shelving area than the second shelving area. The term “different” includes, but is not limited to, different brands, different product forms, different scents, and different manufacturers. The rinse-off care product may be positioned on shelving that is adjacent to or within the first shelving area.

In accordance with another exemplary embodiment, the consumer product line-up comprises a shelving area defined by a lower shelf, an upper shelf, and opposing side shelving locations; a plurality of different leave-on antiperspirant or deodorant products positioned within the shelving area, at least some of the plurality of different leave-on antiperspirant or deodorant products being proximate each of the opposing side shelving locations; and a rinse-off care product also positioned within the shelving area.

Another consumer product line-up embodiment comprises a leave-on antiperspirant or deodorant product positioned for sale at a first location, and a rinse-off care product positioned for sale at a second location that is within a distance (e.g., 1, 2, 3, 4, 5, 6, 7, 8, 9, or 10 feet) of the first location, wherein a consumer is able to purchase the leave-on antiperspirant or deodorant product and the rinse-off care product and use them according to a underarm care regimen.

In accordance with another embodiment, the consumer product line-up comprises a leave-on antiperspirant or deodorant product positioned for sale at a first location, and a rinse-off care product positioned for sale at a second location, wherein both the first location and the second location are visible from a single reference viewpoint.

Yet another consumer product line-up comprises a leave-on antiperspirant or deodorant product comprising first packaging component, and a rinse-off care product comprising second packaging component, wherein the first packaging component and second packaging component are affixed to one another or contained within a third packaging component.

### Method for Managing Underarm Malodor

The present invention is also directed to methods for managing underarm malodor. For example, in one method the following steps are conducted: (a) applying a first consumer product to an underarm and rubbing the same onto the skin of the underarm, the first consumer product comprising a zinc containing material; (b) thereafter rinsing the underarm; and (c) thereafter applying a second consumer product to the underarm without rinsing the same for a period of at least one hour, the second consumer product comprising an antiperspirant active and/or deodorant agent.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1) A consumer product line-up, comprising:
(a) a plurality of different leave-on antiperspirant or deodorant products positioned within a first shelving area;
(b) a plurality of different body wash products positioned within a second shelving area that is separate and distinct from the first shelving area; and
(c) a rinse-off care product positioned on shelving that is closer to the first shelving area than the second shelving area, the rinse-off care product comprising indicium that serves as a product selection guide through an indication that the rinse-off care product is for underarm application to manage body odor.

2) The line-up of claim 1, wherein the rinse-off care product is positioned on shelving adjacent to the first shelving area.

3) The line-up of claim 1, wherein the rinse-off care product is positioned on shelving within the first shelving area.

4) The line-up of claim 1, wherein the rinse-off care product comprises an antimicrobial agent.

5) The line-up of claim 4, wherein the antimicrobial agent comprises a zinc-containing material.

6) The line-up of claim 5, wherein the zinc-containing material comprises basic zinc carbonate.

7) The line-up of claim 5, wherein the zinc-containing material comprises zinc pyrithione.

8) The line-up of claim 1, wherein the rinse-off care product comprises packaging including indicium that communicates application of a leave-on antiperspirant or deodorant product after application of the rinse-off care product.

9) The line-up of claim 1, wherein the rinse-off care product has a common brand name with one of the plurality of different leave-on antiperspirant or deodorant products.

10) The line-up of claim 1, wherein the rinse-off care product and at least one of the plurality of different leave-on antiperspirant or deodorant products are from the same manufacturer.

11) A consumer product line-up, comprising:
(a) a shelving area defined by a lower shelf, an upper shelf, and opposing side shelving locations;
(b) a plurality of different leave-on antiperspirant or deodorant products positioned within the shelving area, at least some of the plurality of different leave-on antiperspirant or deodorant products being proximate each of the opposing side shelving locations; and
(c) a rinse-off care product also positioned within the shelving area.

12) The line-up of claim 11, wherein the rinse-off care product comprises an antimicrobial agent.

13) The line-up of claim 12, wherein the antimicrobial agent comprises a zinc-containing material.

14) A consumer product line-up, comprising:
(a) a leave-on antiperspirant or deodorant product positioned for sale at a first location; and
(b) a rinse-off care product positioned for sale at a second location that is within 10 feet of the first location,
wherein a consumer is able to purchase the leave-on antiperspirant or deodorant product and the rinse-off care product and use them according to a underarm care regimen.

15) The line-up of claim 14, wherein the second location is within 5 of the first location.

16) The line-up of claim 14, wherein the second location is adjacent the first location.

17) The line-up of claim 14, wherein the rinse-off care product comprises an antimicrobial agent.

18) The line-up of claim 14, wherein the rinse-off care product comprises indicium that communicates application of the leave-on antiperspirant or deodorant product after application of the rinse-off care product.

19) The line-up of claim 14, wherein the rinse-off care product comprises indicium that communicates application of the rinse-off care product to one's underarms.

20) The line-up of claim 14, wherein the leave-on antiperspirant or deodorant product and the rinse-off care product are from the same manufacturer.

21) The line-up of claim 14, wherein the leave-on antiperspirant or deodorant product and the rinse-off care product share a brand name.

22) The line-up of claim 14, wherein the antiperspirant or deodorant product comprises an aluminum or aluminum-zirconium antiperspirant active, and wherein the rinse-off care product comprises a zinc containing material.

23) A consumer product line-up, comprising:
(a) a leave-on antiperspirant or deodorant product positioned for sale at a first location; and
(b) a rinse-off care product positioned for sale at a second location,
wherein both the first location and the second location are visible from a single reference point of view.

24) A consumer product line-up, comprising:
(a) a leave-on antiperspirant or deodorant product comprising first packaging component; and
(b) a rinse-off care product comprising second packaging component,
wherein the first packaging component and second packaging component are affixed to one another or contained within a third packaging component.

25) A method for managing underarm malodor, the method comprising the steps of:
(a) applying a first consumer product to an underarm and rubbing the same onto the skin of the underarm, the first consumer product comprising a liquid carrier material and an antimicrobial agent, the antimicrobial agent being in the form of solid particles within the liquid carrier material;
(b) thereafter rinsing the underarm; and
(c) thereafter applying a second consumer product to the underarm without rinsing the same for a period of at least one hour, the second consumer product comprising an antiperspirant active and/or deodorant agent.

26) The method of claim 25, wherein the antimicrobial agent has an MIC value of 2500 μg/ml or less against at least one strain of at least one of staphylococcus epidermidis and corynebacterium mucifaciens.

27) The method of claim 25, wherein the antimicrobial agent has an MIC value of 1000 μg/ml or less against at least one strain of at least one of staphylococcus epidermidis and corynebacterium mucifaciens.

28) The method of claim 25, wherein the antimicrobial agent has an MIC value of 500 μg/ml or less against at least one strain of at least one of staphylococcus epidermidis and corynebacterium mucifaciens.

29) The method of claim 25, wherein the antimicrobial agent has an MIC value of 100 μg/ml or less against at least one strain of at least one of staphylococcus epidermidis and corynebacterium mucifaciens.

30) The method of claim 25, wherein at least some of the solid particles have a particle size of from about 0.5 μm to about 100 μm.

31) The method of claim 25, wherein at least some of the solid particles have a particle size of from about 0.5 μm to about 50 μm.

32) A method for managing underarm malodor, the method comprising the steps of:
(a) applying a first consumer product to an underarm and rubbing the same onto the skin of the underarm, the first consumer product comprising a zinc containing material;
(b) thereafter rinsing the underarm; and
(c) thereafter applying a second consumer product to the underarm without rinsing the same for a period of at least one hour, the second consumer product comprising an antiperspirant active and/or deodorant agent.

33) The method of claim 32, wherein the zinc-containing material comprises zinc carbonate, zinc pyrithione, or mixtures thereof.

34) A rinse-off care product, comprising:
(a) a container;
(b) a composition disposed within the container, the composition comprising a liquid carrier material and an antimicrobial agent, the antimicrobial agent being in the form of solid particles within the composition, at least some of the solid particles being sized for delivery into underarm hair follicles;
(c) a first indicium associated with the container that serves as a product selection guide through an indication that the rinse-off care product is for underarm application to manage body odor; and
(d) a second indicium associated with the container that instructs a user to apply the composition to an underarm, followed by a rinse step.

35) A rinse-off care product, comprising:
(a) a container;
(b) a composition disposed within the container, the composition comprising a liquid carrier material and an antimicrobial agent, the antimicrobial agent being in the form of solid particles within the composition and having an MIC value of 2500 μg/ml or less against at least one strain of at least one of staphylococcus epidermidis and corynebacterium mucifaciens;
(c) a first indicium associated with the container that serves as a product selection guide through an indication that the rinse-off care product is for underarm application to manage body odor; and
(d) a second indicium associated with the container that instructs a user to apply the composition to an underarm, followed by a rinse step.

36) The rinse-off care product of claim 35, wherein the antimicrobial agent has an MIC value of 1000 μg/ml or less against at least one strain of at least one of staphylococcus epidermidis and corynebacterium mucifaciens.

37) The rinse-off care product of claim 35, wherein the antimicrobial agent has an MIC value of 100 μg/ml or less against at least one strain of at least one of staphylococcus epidermidis and corynebacterium mucifaciens.

38) The rinse-off care product of claim 35, wherein the solid particles comprise particles having a particle size of from about 0.5 μm to about 100 μm.

39) The rinse-off care product of claim 35, wherein the solid particles comprise particles having a particle size of from about 0.5 μm to about 50 μm.

40) The rinse-off care product of claim 35, wherein the antimicrobial agent comprises a zinc containing material.

41) A rinse-off care product, comprising:
(a) a container;
(b) a composition disposed within the container, the composition comprising a liquid carrier material and an antimicrobial agent;
(c) a first indicium associated with the container that serves as a product selection guide through an indication that the rinse-off care product is for underarm application to manage body odor;
(d) a second indicium associated with the container that instructs a user to apply the composition to an underarm, followed by a rinse step; and
(e) a third indicium associated with the container that indicates application of a leave-on antiperspirant or deodorant product after the rinse-off care product is used.

42) The rinse-off care product of claim 41, wherein the antimicrobial agent is in the form of solid particles within the composition and upon application to the skin.

43) The rinse-off care product of claim 42, wherein the solid particles have an MIC value of 2500 μg/ml or less against at least one strain of at least one of staphylococcus epidermidis and corynebacterium mucifaciens.

44) The rinse-off care product of claim 42, wherein at least some of the solid particles have a particle size of from about 0.5 μm to about 100 μm.

45) A rinse-off care product, comprising:
(a) a container;
(b) a composition disposed within the container, the composition comprising a liquid carrier material and an antimicrobial agent;
(c) a first indicium associated with the container that serves as a product selection guide through an indication that the rinse-off care product is for underarm application to manage body odor;
(d) a second indicium associated with the container that instructs a user to apply the composition to an underarm, followed by a rinse step; and
(e) a third indicium associated with the container that indicates that the rinse-off care product can be used in place of a leave-on antiperspirant or deodorant product.
